(19) United States
(12) Patent Application Publication
(10) Pub. No.: US 2024/0148686 A1
(43) Pub. Date: May 9, 2024

(54) PHARMACEUTICAL COMPOSITION OF FORSKOLIN-ISOFORSKOLIN AND PENTACYCLIC TRITERPENOID COMPOUND, AND APPLICATION THEREOF

(71) Applicant: CHINA PHARMACEUTICAL UNIVERSITY, Jiangsu (CN)

(72) Inventor: Liang WU, Jiangsu (CN)

(73) Assignee: CHINA PHARMACEUTICAL UNIVERSITY, Jiangsu (CN)

(21) Appl. No.: 18/278,628

(22) PCT Filed: Apr. 26, 2021
(86) PCT No.: PCT/CN2021/090005

§ 371 Date: Oct. 30, 2023

(30) Foreign Application Priority Data
Feb. 24, 2021 (CN) 202110206310.9

(51) Int. Cl. A61K 31/352; A61K 31/56; A61P 1/16

(52) U.S. Cl. CPC A61K 31/352; A61K 31/56; A61P 1/16

(57) ABSTRACT

The present disclosure relates to a pharmaceutical composition, including a first component and a second component, where the first component is selected from the group consisting of forskolin, isoforskolin, and a pharmaceutically acceptable salt thereof; and the second component is selected from the group consisting of a pentacyclic triterpenoid and a pharmaceutically acceptable salt thereof. The pharmaceutical composition of the present disclosure allows the combined use of forskolin-isoforskolin with a pentacyclic triterpenoid. Compared with the use of each of the two components alone, the combined use can play a significant synergistic role in prevention and treatment of a metabolic disease, a fibrotic disease, and a liver disease. Therefore, the pharmaceutical composition of the present disclosure has promising medicinal prospects in treatment, prevention, and improvement of obesity, a non-alcoholic fatty liver disease (NAFLD), liver damage, liver fibrosis, and the like.

### TECHNICAL FIELD

The present disclosure belongs to the field of medicine, and relates to a pharmaceutical composition including forskolin-isoforskolin and a pentacyclic triterpenoid, and a use of the pharmaceutical composition in preparation of a drug for preventing, improving, or treating a metabolic disease, a fibrotic disease, and a liver disease.

### BACKGROUND ART

With the improvement of an economic level, a diet structure of people has undergone a huge change with an intake of lipids increased, and an obesity population has gradually been expanded. The World Health Organization (WHO) defines people with a body mass index (BMI) larger than or equal to 25 as overweight people. Obesity can cause a variety of metabolic diseases, such as diabetes, hypertension, hyperlipidemia, and fatty liver. Non-alcoholic fatty liver disease (NAFLD) is a disease mainly characterized by lipid accumulation and degeneration in liver cells. A pathological section of NAFLD mainly shows a significant increase in lipid droplets in liver cells, inflammation, and necrosis, which may progress into liver damage/liver fibrosis. With the rapid change of a life style, the development tendency of NAFLD in China is increasing, which has become a major public health issue. Data show that a prevalence rate of NAFLD in China is 29.2%, and there is a heavy burden of NAFLD in middle-aged people, men, northwest China, Taiwan of China, and areas with a gross domestic product (GDP) per capita of more than 100,000 yuan. People pay less attention to liver diseases than to diabetes and hypertension. Both research results and clinical results prove that viral hepatitis, drug-induced liver injury (DILI), fatty liver, or the like may progress into liver fibrosis, which is mainly attributed to deposition of extracellular matrix (ECM) of liver cells; and after the viral hepatitis, DILI, fatty liver, or the like progresses into liver cirrhosis and liver cancer, a mortality rate is extremely high. There is still a lack of effective drugs for treating liver cirrhosis and liver cancer, and a 5-year survival rate of liver cirrhosis and liver cancer patients undergoing hepatectomy is about 50% to 60%. However, the current common means for treating obesity and fatty liver is restricted diet+moderate exercise, and there are very few drugs for treating obesity and fatty liver clinically. In order to reverse this pathological process, the development of relevant drugs has become a top priority.

Forskolin is a compound (also known as Coleus barbatus extract) extracted from Coleus barbatus of the Coleus genus of the mint family. Forskolin has a chemical formula of C₂₂H₃₄O₇, with CAS: 66575-29-9. A structural formula of forskolin is as follows:

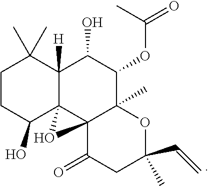 [SMILES: C=C[C@@]1(C)CC(=O)[C@]23O[C@@]24[C@@H](O)CCC(C)(C)[C@@H]4[C@H](O)[C@H](OC(C)=O)[C@@]3(C)O1]

Isoforskolin has a chemical formula of C₂₂H₃₄O₇, with CAS: 64657-21-2, and is an isomer of forskolin. A structural formula of isoforskolin is as follows:

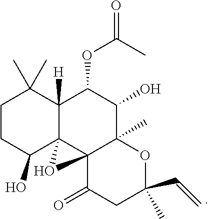 [SMILES: C=C[C@@]1(C)CC(=O)[C@]23O[C@@]24[C@@H](O)CCC(C)(C)[C@@H]4[C@H](OC(C)=O)[C@H](O)[C@@]3(C)O1]

Pharmacodynamic results show that forskolin, as a potent adenylate cyclase activator, can increase a level of cAMP to participate in the regulation of various cell functions and effectively promote the differentiation of neurons and the growth and development of nerves in a central nervous system (CNS) and a peripheral nervous system (PNS), thereby producing important effects on a cardiovascular system (CVS), a respiratory system, a tumor, or the like; and forskolin has pharmacological actions such as cardiotonic action, asthma relief, anti-tumor action, anti-thrombotic action, and intraocular pressure (IOP) reduction. Isoforskolin, as an isomer of forskolin, also has a variety of similar pharmacological actions to forskolin. In recent years, with the progress of research, it has been found that forskolin and isoforskolin also have effects such as antidepression and weight loss. Clinically, forskolin and isoforskolin have begun to be used for treating cardiovascular diseases (CVDs), tumors, common diseases in the elderly, or the like. Forskolin can play a role in weight loss and fatty liver treatment by promoting the hydrolysis and β-oxidation of triglycerides (TGs), and can also play a role of anti-fibrosis by activating a PKA signaling pathway and reducing oxidative stress and inflammation.

Pentacyclic triterpenoids are a group of triterpenoids that each are produced by linking six isoprene units with five closed rings as a parent, and are mainly divided into four categories: oleanane, ursane, lupane, and friedelane. A previous major research direction of pentacyclic triterpenoids is an anti-tumor effect, and many active compounds are screened out as anti-tumor drugs. Anti-tumor effects of these active compounds exhibit characteristics of multiple sites, multiple links, and multiple targets, which can not only allow the active compounds to continuously act for a long time, but also prolong a survival time of a tumor patient. Therefore, these active compounds are expected to become a new generation of anti-tumor drugs. In addition, pentacyclic triterpenoids have anti-inflammatory and antibacterial functions and hepatoprotective and choleretic effects. Pentacyclic triterpenoids play a role in weight loss and fatty liver treatment by promoting the secretion of leptin and the hydrolysis of TG. Pentacyclic triterpenoids can inhibit signaling pathways such as NF-κB to play an anti-inflammatory role, reduce an inflammatory response, scavenge free radicals, and inhibit the proliferation of collagen fibers, thereby alleviating liver damage and liver fibrosis.

Asiaticoside is a pentacyclic triterpenoid saponin extracted from Centella asiatica, and can promote the local hydration and collagen production, regulate the proliferation of fibroblasts, play an antioxidant role, reduce the inflammation of a wound, stimulate the maturation of a scar, and promote the healing of a wound. At present, asiaticoside is widely used for treating various skin diseases and repairing a wound clinically, and is increasingly used in various skin care products. Asiaticoside has a molecular formula of C₄₈H₇₈O₁₉, with CAS: 16830-15-2. A structural formula of asiaticoside is as follows:

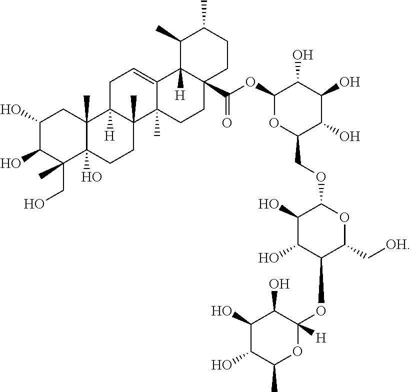

Asiatic acid is an ursane pentacyclic triterpenic acid mainly derived from Centella asiatica of the parsley family. Asiatic acid is a Centella asiatica extract, and has a wide range of pharmacological activities, such as treatment of skin trauma and chronic ulcer, antidepression, prevention and treatment of CVDs, anti-Parkinson's disease (PD), and anti-malignant tumors, among which the anti-tumor activity is particularly significant. Asiatic acid has a molecular formula of C₃₀H₄₈O₅, with CAS: 464-92-6. A structural formula of asiatic acid is as follows:

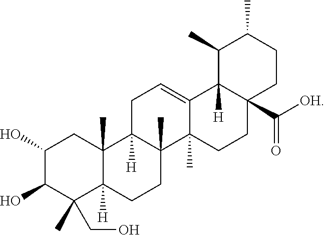

Oleanolic acid (OA) is a pentacyclic triterpenoid extracted from a fruit of Swertia mileensis of the Swertia genus of the Gentianaceae family or Ligustrum lucidum, and exists in a variety of plants as free bodies and glucosides. OLA is clinically used for liver protection and enzyme reduction, and treatment of bronchitis, pneumonia, acute tonsillitis, periodontitis, bacillary dysentery, acute gastroenteritis, and urinary tract infection (UTI). OLA has a chemical formula of C₃₀H₄₈O₃, with CAS: 508-02-1. A structural formula of OLA is as follows:

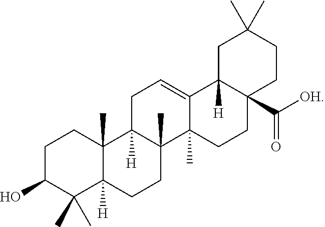

Betulinic acid, also known as betulic acid, is a lupane pentacyclic triterpenoid, which widely exists in the nature and is mainly derived from birch bark. In recent years, scientific research has shown that betulinic acid is a very valuable natural product; and betulinic acid and derivatives thereof exhibit a great potential as biological agents in anti-cancer and other aspects, and play an important role in anti-oxidative damage and immunoregulation processes of a body. Betulinic acid has a chemical formula of C₃₀H₄₈O₃, with CAS: 472-15-1. A structural formula of betulinic acid is as follows:

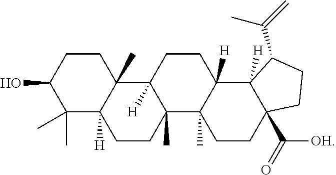

The use of a combination of forskolin-isoforskolin with a pentacyclic triterpenoid in prevention and treatment of a metabolic disease, a fibrotic disease, and a liver disease has not been reported.

### SUMMARY OF THE INVENTION

To solve the problems in the prior art, the present disclosure provides a pharmaceutical composition, the composition includes two components, where a first component is selected from the group consisting of forskolin, isoforskolin, and a pharmaceutically acceptable salt thereof; and a second component is selected from the group consisting of a pentacyclic triterpenoid and a pharmaceutically acceptable salt thereof.

According to an embodiment of the present disclosure, the pentacyclic triterpenoid is one or more selected from the group consisting of an oleanane pentacyclic triterpenoid, an ursane pentacyclic triterpenoid, a lupane pentacyclic triterpenoid, and a friedelane pentacyclic triterpenoid. Preferably, the pentacyclic triterpenoid is one or more selected from the group consisting of an oleanane pentacyclic triterpenoid, an ursane pentacyclic triterpenoid, and a lupane pentacyclic triterpenoid. The oleanane pentacyclic triterpenoid is OA; the ursane pentacyclic triterpenoid is selected from the group consisting of asiatic acid and analogues thereof, such as asiatic acid, madecassic acid, asiaticoside, and madecassoside; and the lupane pentacyclic triterpenoid is selected from the group consisting of betulinic acid and betulinol.

According to an embodiment of the present disclosure, the first component may be one, or a mixture of two or more, selected from the group consisting of forskolin, isoforskolin, a pharmaceutically acceptable salt of forskolin, and a pharmaceutically acceptable salt of isoforskolin.

According to an embodiment of the present disclosure, a weight ratio of the first component to the second component is (100:1) to (1:100), preferably (50:1) to (1:50), and more preferably (20:1) to (1:20), for example, the weight ratio may be selected from the group consisting of 1:1, 2:1, 3:1, 4:1, 5:1, 6:1, 7:1, 8:1, 9:1, 10:1, 1:2, 1:3, 1:4, 1:5, 1:6, 1:7, 1:8, 1:9, and 1:10.

In some embodiments of the present disclosure, the pharmaceutical composition includes only the first component and the second component as active components; and in some other embodiments, the pharmaceutical composition further includes an additional active component.

According to an embodiment of the present disclosure, the pharmaceutical composition further includes a pharmaceutically acceptable adjuvant. The pharmaceutically acceptable adjuvant is an excipient, a binder, a disintegrating agent, an emulsifying agent, a preservative, a suspending agent, a fragrance, a pH adjusting agent, a flocculating agent and a deflocculating agent, a surfactant, a filler, a lubricant, a thickening agent, a humectant, a plasticizer, a bacteriostatic agent, a coating material, a foaming agent, a defoaming agent, an encapsulation agent, an isoosmotic adjusting agent, or a stabilizing agent.

The present disclosure also provides a formulation including the pharmaceutical composition described above, where a dosage form of the formulation is selected from the group consisting of an oral dosage form such as a tablet, an effervescent tablet, a nebulizer, a gel, a granule, a pill, a capsule, a dropping pill, a suspension, and an injection, and is preferably an oral dosage form.

Further, a mass of the pharmaceutical composition of the present disclosure is 1% to 99% and preferably 1% to 60% of a total mass of the pharmaceutical formulation.

The present disclosure also provides a use of the pharmaceutical composition described above in preparation of a drug for preventing, treating, or improving a metabolic disease, a fibrotic disease, and a liver disease.

According to an embodiment of the present disclosure, the metabolic disease, the fibrotic disease, and the liver disease are selected from the group consisting of hyperglycaemia, hypertriglyceridemia (HTG), hypercholesterolemia, diabetes, obesity, excess visceral fat accumulation, CVD, atherosclerosis (AS), liver fibrosis, renal fibrosis, pulmonary fibrosis, skin fibrosis, cardiac fibrosis, fatty liver, liver damage, liver cirrhosis, chronic hepatitis, liver cancer, cholestasis, and cholelithiasis; and the metabolic disease, the fibrotic disease, and the liver disease are preferably selected from the group consisting of obesity, NAFLD, liver damage, and liver fibrosis.

### Beneficial Effects

1) The pharmaceutical composition of the present disclosure allows the combined use of forskolin-isoforskolin with a pentacyclic triterpenoid. Compared with the use of each of the two components alone, the combined use of forskolin-isoforskolin with a pentacyclic triterpenoid can play a significant synergistic role in prevention and treatment of a metabolic disease, a fibrotic disease, and a liver disease. It has been verified through experiments that, for obese mice induced by a high-fat diet, the pharmaceutical composition of the present disclosure can significantly reduce a body weight of mice, alleviate the fat accumulation in the liver and other internal organs, and reduce the free fatty acid (FFA) and total cholesterol (TC) levels in serum. In addition, in a CCl₄-induced liver damage/liver fibrosis mouse experiment, Sirius red staining results show that a collagen content in the liver of mice in the combined administration group is significantly lower than a collagen content in the liver of unadministered mice, and the combined administration has a significant synergistic effect. Hematoxylin and eosin (H&E) staining results show that liver cells of mice in the combined administration group are arranged more regularly than liver cells of mice in the unadministered group, and contents of aspartate aminotransferase (AST) and alanine aminotransferase (ALT) in the liver are significantly reduced in the combined administration group, indicating that the combined use of forskolin-isoforskolin with a pentacyclic triterpenoid can significantly reduce the liver damage, improve the liver function, and inhibit the liver fibrosis.

2) The pharmaceutical composition of the present disclosure allows the combined use of two known natural active components that have been medicinally developed, without obvious toxic and side effects; and the pharmaceutical composition has a wide range of application values.

### BRIEF DESCRIPTION OF THE DRAWINGS

FIG. 1 shows an impact of the pharmaceutical composition on a body weight of an NAFLD model mouse.

FIG. 2 shows an impact of the pharmaceutical composition on a liver weight/body weight ratio of an NAFLD model mouse.

FIG. 3 shows an impact of the pharmaceutical composition on a mass of inguinal white fat of an NAFLD model mouse.

FIG. 4 shows oil red O staining results of liver sections of an NAFLD model mouse administered with the pharmaceutical composition.

FIG. 5 shows an impact of the pharmaceutical composition on an oil red O-stained area of a liver section of an NAFLD model mouse.

FIG. 6 shows an impact of the pharmaceutical composition on a liver weight/body weight ratio of a liver fibrosis model mouse.

FIG. 7 shows a type III precollagen (PCIII) content in a liver of a liver fibrosis model mouse administered with the pharmaceutical composition.

FIG. 8 shows H&E staining results of liver sections of a liver fibrosis model mouse administered with the pharmaceutical composition.

FIG. 9 shows Sirius red staining results of liver sections of a liver fibrosis model mouse administered with the pharmaceutical composition.

FIG. 10 shows an impact of the pharmaceutical composition on a Sirius red-stained area of a liver section of a liver fibrosis model mouse.

### DETAILED DESCRIPTION OF THE EMBODIMENTS

The technical solutions of the present disclosure will be further described in detail below in conjunction with specific examples. It should be understood that the following examples are merely intended to exemplarily illustrate and explain the present disclosure, and should not be interpreted as a limitation to the protection scope of the present disclosure. All technologies implemented based on the content of the present disclosure fall within the protection scope of the present disclosure.

Unless otherwise specified, the raw materials and reagents used in the following examples are commercially-available products, or may be prepared by known methods.

Notes: Statistical analysis is conducted by the GraphPad Prism 8.0 statistical software, and statistical analysis results are analyzed by t-Test. Compared with a model group, *P<0.05 indicates a statistically-significant difference between the groups: **P<0.01, ****P<0.001, and ****P<0.0001. A calculation method of a combined index (CI) is as follows: according to a Burgi's method, a CI value is determined based on the following formulas: q=E_{a/2+b/2}/Eₐ (when Eₐ>E_{b}) or q=E_{a/2+b/2}/E_{b} (when Eₐ<E_{b}), where Eₐ, E_{b}, and E_{a/2+b/2} represent effects of a drug a group, a drug b group, and a half-dose drug a+half-dose drug b group, respectively; q<1 indicates that the combined use of the two drugs produces an antagonistic effect; q=1 indicates that the combined use of the two drugs produces an additive effect; and q>1 indicates that the combined use of the two drugs produces a synergistic effect. In short, if an effect of the half-dose drug a+half-dose drug b group is stronger than an effect of the superior single drug group, it indicates that the combined use has a synergistic effect.

### Example 1 Therapeutic Effects of the Pharmaceutical Composition of the Present Disclosure on an Obesity Model and a Fatty Liver Model Induced by a High-Fat Diet

Therapeutic effects of the combined administration of forskolin-isoforskolin with a pentacyclic triterpenoid on obesity and fatty liver models induced by a high-fat diet were compared with therapeutic effects of the administration of each component alone on the obesity and fatty liver models induced by the high-fat diet to verify a synergistic therapeutic effect of the pharmaceutical composition of the present disclosure.

1. Establishment of NAFLD Model Mice

160 SPF-grade male C57 mice at 8 weeks to 9 weeks were adaptively raised for one week, and then 140 obese mice with a consistent body weight were selected and evenly divided into 14 groups with 10 mice per group, including a model group (M group), a forskolin group (F group), an isoforskolin group (IF group), an asiatic acid group (AA group), an asiaticoside group (AS group), an OA group (OA group), a betulinic acid group (BA group), a forskolin+asiatic acid combined administration group (F+AA group), a forskolin+asiaticoside combined administration group (F+AS group), forskolin+OA combined administration groups (low, medium, and high) (F+OA-1:4, 1:1, and 4:1 groups), a forskolin+betulinic acid combined administration group (F+BA group), and an isoforskolin+OA combined administration group (IF+OA group). The mice were fed by a high-fat diet (including 35% of fat, 26% of carbohydrates, and 26% of proteins) continuously for 12 weeks to establish obesity and fatty liver models, and during the modeling, a drug was intragastrically administered continuously for 12 weeks. The experimental animals were raised in ordinary mouse cages in an environment with a room temperature of 24±2° C., a humidity of 40% to 60%, and a 12 h light/12 h dark cycle.

2. Treatment Experiment of NAFLD Model Mice

Administration Dose

| Group | Dose (mg/kg) |
|---|---|
| M | — |
| F | 20 |
| IF | |
| AA | |
| AS | |
| OA | |
| BA | |
| F + AA | 10 + 10 |
| F + AS | |
| F + OA-1:4 | 4 + 16 |
| F + OA-1:1 | 10 + 10 |
| F + OA-4:1 | 16 + 4 |
| F + BA | 10 + 10 |
| IF + OA | |

Administration mode: A drug was prepared with a 0.5% CMC-Na solution as a solvent into a suspension, and the suspension was intragastrically administered once a day continuously for 12 weeks.

Sampling and Sample Storage:

The mice were administered with a drug continuously for 12 weeks, and after the last administration, the mice were fasted without water deprivation for 12 h; a body weight of each mouse was measured, eyeballs were removed, blood was collected, and a liver was taken out and weighed; a part of the liver tissue was stored in 4% paraformaldehyde (PFA), and another part of the liver tissue was stored in liquid nitrogen; and inguinal fat was collected from a right thigh of each mouse, weighed, and stored in liquid nitrogen.

A blood sample was allowed to stand in a 4° C. refrigerator for 4 h and then centrifuged at 3,500 rpm for 10 min to 15 min; and a resulting supernatant was collected and tested by a blood bio-chemical analyzer to determine the FFA and TC levels in serum. The liver tissue stored in 4% PFA was frozen and sectioned, and then stained with oil red O to obtain slide specimens. The liver tissue stored in liquid nitrogen was homogenized and then centrifuged, and a resulting supernatant was collected and then tested by a kit to determine a TG content.

3. Experimental Results

1) Body Weight Changes

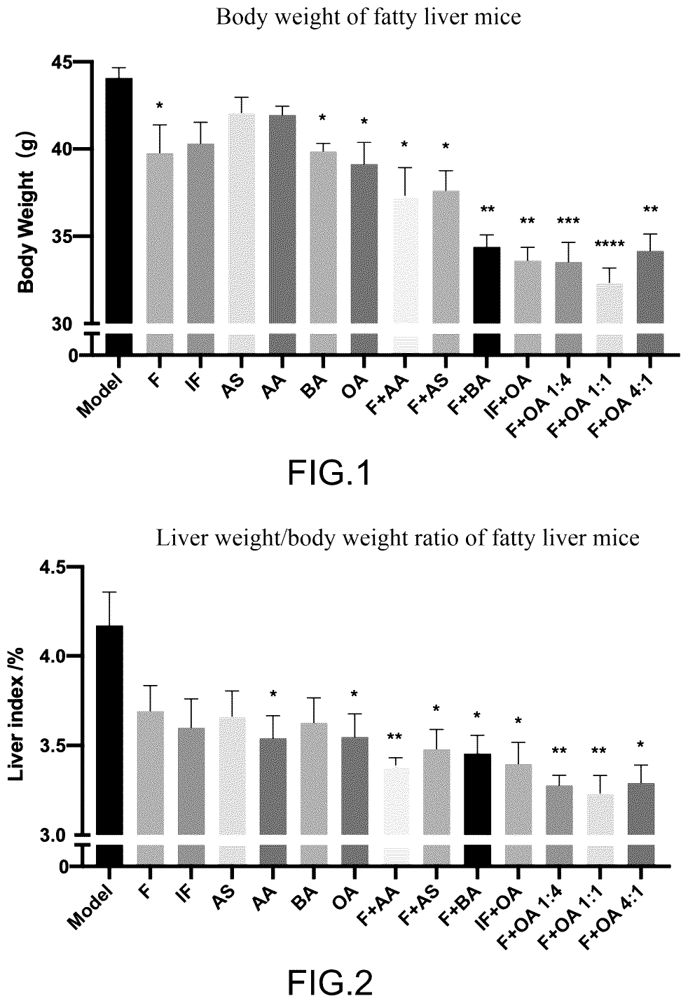

Results are shown in FIG. 1. A body weight of mice in the model group increases rapidly. A body weight of mice in the forskolin+OA (1:1) combined administration group increases most slowly, and secondly, a body weight of mice in the isoforskolin+OA combined administration group also increases slowly, indicating that forskolin and isoforskolin can inhibit the increase of a body weight of mice similarly. The increase of a body weight of mice in the remaining combined administration group can also be inhibited. A body weight of mice in each combined administration group is lower than a body weight of mice in each single administration group, and a CI value of a body weight reduction rate is greater than 1. At a same dose, a drug in each single administration group exhibits a specified effect of inhibiting the increase of a body weight, but the effect is limited; and an effect of inhibiting the increase of a body weight of mice in a combined administration group is significantly stronger than an effect of inhibiting the increase of a body weight of mice in a single administration group, indicating that the combined use of forskolin-isoforskolin with a pentacyclic triterpenoid has a synergistic weight loss effect.

2) Liver Index

NAFLD is characterized by fat accumulation in the liver, and involves an increased liver weight and an increased liver index (liver index=liver weight/body weight*100). A therapeutic effect of a drug for fatty liver in mice can be evaluated through a liver index. Results are shown in FIG. 2. For the fatty liver models induced by the high-fat diet, the forskolin+OA (1:1) combined administration group has the lowest liver index, and other combined administration groups also have reduced liver indexes, which do not have a significant difference; and a liver index of the liver of mice in a combined administration group is significantly lower than a liver index of the liver of mice in the single administration group, and a CI value of a liver index reduction rate is greater than 1. At a same dose, the single administration can reduce a liver index to some extent, and an effect of reducing a liver index of mice in a combined administration group is stronger than an effect of reducing a liver index of mice in a single administration group, indicating that the combined use of forskolin-isoforskolin with a pentacyclic triterpenoid has a synergistic effect.

3) Visceral Fat

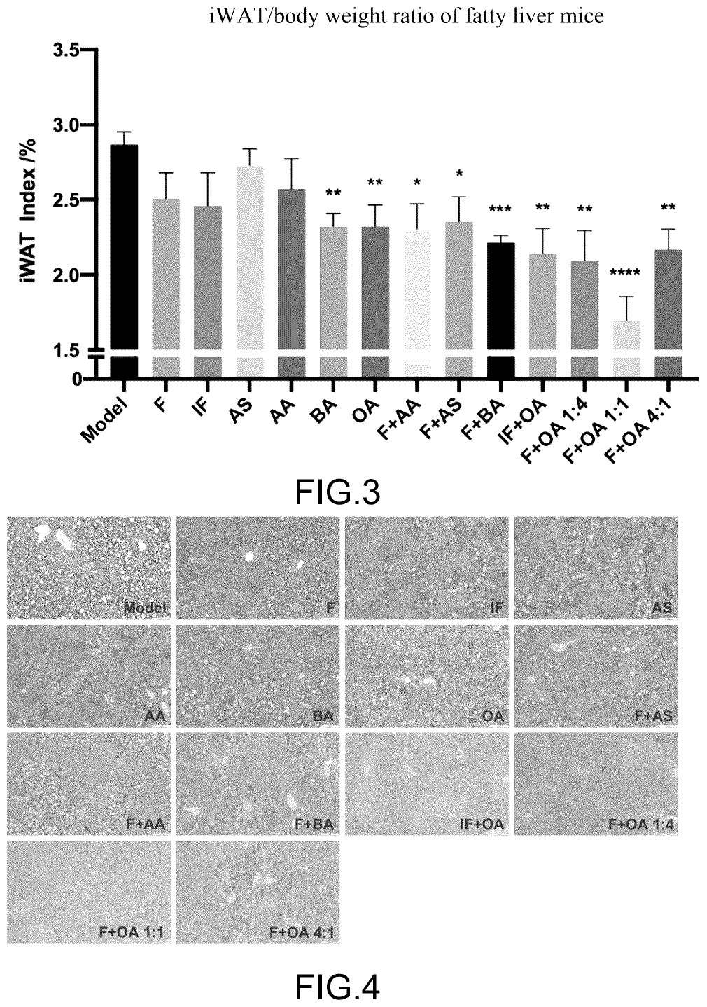

Visceral fat is distributed on a surface of each organ, where inguinal fat is the most typical and can be easily collected. A content of inguinal fat can reflect a content of visceral fat to some extent, and a ratio of a weight of inguinal fat to a body weight is called a fat weight/body weight ratio. Results are shown in FIG. 3. Compared with a fat weight/body weight ratio of mice in the model group, the forskolin+OA (1:1) combined administration group has the lowest fat weight/body weight ratio, and in other combined administration groups, the fat weight/body weight ratio is also significantly reduced; and a fat weight/body weight ratio in a combined administration group is significantly lower than a fat weight/body weight ratio in a single administration group, and a CI value of fat weight/body weight reduction efficiency is greater than 1. At a same dose, the single administration can reduce a liver index to some degree, and an effect of reducing a liver index of mice in a combined administration group is stronger than an effect of reducing a liver index of mice in a single administration group, indicating that the combined use of forskolin-isoforskolin with a pentacyclic triterpenoid has a synergistic visceral fat reduction effect.

4) Blood Indexes

A concentration of FFAs in serum is related to lipid metabolism, glucose metabolism, and endocrine functions, and a concentration of FFAs will increase due to a disease such as diabetes, hepatic dysfunction, or obesity. The liver is a major organ for synthesis and storage of cholesterol, and a serum concentration of cholesterol can serve as an index for lipid metabolism. When fatty liver is produced due to obesity, a TC content in serum will increase, and a high TC content will lead to AS and cardiac diseases in a patient. Results are shown in Table 1. Compared with FFA and TC levels in serum of mice in the model group, the forskolin+OA (1:1) combined administration group has the lowest FFA and TC levels, and in other combined administration groups, the FFA and TC levels are also significantly reduced; and FFA and TC levels in a combined administration group are significantly lower than FFA and TC levels in a single administration group on average, and CI values of serum FFA and TC level reduction rates are greater than 1. At a same dose, the single administration can reduce serum FFA and TC levels to some extent, and an effect of reducing serum FFA and TC levels of mice in a combined administration group is stronger than an effect of reducing serum FFA and TC levels of mice in a single administration group, indicating that the combined use of forskolin-isoforskolin with a pentacyclic triterpenoid has a synergistic serum FFA and TC level reduction effect.

Table 1 Impact of each administration on serum FFA and TC contents in fatty liver model mice (mmol/L, x̅±s)

| Group | FFA | TC |
|---|---|---|
| M | 1.523 ± 0.132 | 9.76 ± 0.74 |
| F | 1.324 ± 0.124 | 7.44 ± 0.86 |
| IF | 1.334 ± 0.153 | 7.24 ± 0.64 |
| AA | 1.286 ± 0.143 | 7.26 ± 0.74 |
| AS | 1.238 ± 0.186* | 7.09 ± 0.34 |
| OA | 1.182 ± 0.113* | 6.84 ± 0.54* |
| BA | 1.245 ± 0.201 | 7.02 ± 0.63 |
| F + AA | 1.064 ± 0.084** | 6.25 ± 0.57** |
| F + AS | 1.038 ± 0.095** | 6.40 ± 0.24** |
| F + BA | 0.965 ± 0.107** | 6.23 ± 0.52** |
| IF + OA | 0.949 ± 0.097** | 5.81 ± 0.41** |
| F + OA 1:4 | 0.949 ± 0.086** | 5.56 ± 0.42*** |
| F + OA 1:1 | 0.928 ± 0.067*** | 5.42 ± 0.24*** |
| F + OA 4:1 | 0.963 ± 0.087*** | 5.86 ± 0.52** |

5) Hepatic TG Content

In NAFLD liver cells, a TG content increases, and TG cannot undergo normal esterolysis and β-oxidation. A TG content in the liver of mice can be evaluated to reflect an effect of a drug. Results are shown in Table 2. For the fatty liver models induced by the high-fat diet, compared with the model group, a drug in each administration group can reduce a TG content, where the forskolin+OA (1:1) combined administration group has the lowest TG content, the isoforskolin+OA combined administration group has a medium TG content higher only than the lowest TG content, and in other combined administration groups, a TG content can also be significantly reduced; and a CI value of a hepatic TG reduction rate is greater than 1. At a same dose, the single administration can reduce hepatic TGs to some degree, and an effect of reducing a hepatic TG content of mice in a combined administration group is stronger than an effect of reducing a hepatic TG content of mice in a single administration group, indicating that the combined use of forskolin-isoforskolin with a pentacyclic triterpenoid has a synergistic effect.

Table 2 Impact of each administration on a hepatic TG content in fatty liver model mice (mmol/L, x̅±s)

| Group | TG |
|---|---|
| M | 1.32 ± 0.07 |
| F | 1.01 ± 0.07* |
| IF | 1.09 ± 0.08* |
| AA | 1.17 ± 0.12 |
| AS | 1.18 ± 0.08 |
| OA | 1.03 ± 0.09* |
| BA | 1.10 ± 0.08 |
| F + AA | 0.87 ± 0.08** |
| F + AS | 0.88 ± 0.10** |
| F + BA | 0.89 ± 0.07** |
| IF + OA | 0.84 ± 0.09** |
| F + OA 1:4 | 0.85 ± 0.08** |
| F + OA 1:1 | 0.82 ± 0.08** |
| F + OA 4:1 | 0.87 ± 0.11** |

6) Oil Red O Staining and Quantification

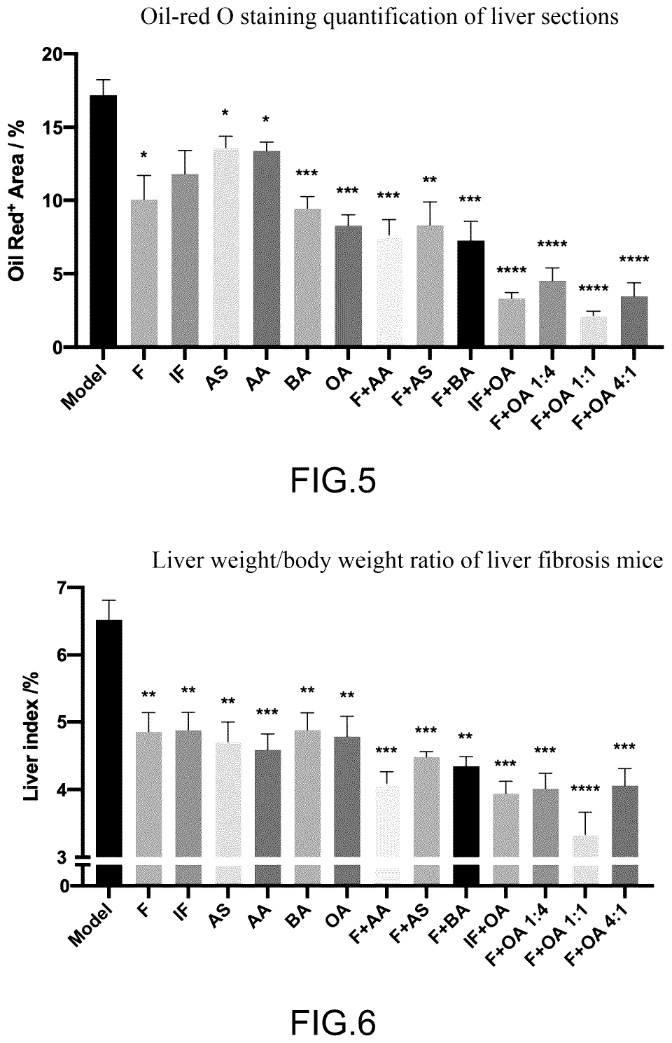

To reveal fat in a tissue, the tissue is often stained with oil red O, and an area stained red represents lipid droplets in the liver, such that a fat level in the liver can be quantified. Results are shown in FIG. 4 and FIG. 5. For the fatty liver models induced by the high-fat diet, the model group has a large area stained red; and compared with the model group, a drug in each administration group can reduce an area stained red, where the forskolin+OA (1:1) combined administration group has the smallest area stained red, the isoforskolin+OA combined administration group has a medium area stained red larger only than the smallest area, and in other combined administration groups, an area stained red can also be significantly reduced. At a same dose, the single administration can also reduce a hepatic fat content, and an effect of reducing a hepatic fat content in a combined administration group is stronger than an effect of reducing a hepatic fat content in a single administration group, indicating that the combined use of forskolin-isoforskolin with a pentacyclic triterpenoid has a synergistic effect.

### Example 2 Therapeutic Effects of the Pharmaceutical Composition of the Present Disclosure on a Chronic Liver Fibrosis Model Induced by CCl₄

A therapeutic effect of the combined administration of forskolin with a pentacyclic triterpenoid on the chronic liver fibrosis model induced by CCl₄ was compared with a therapeutic effect of the administration of each component alone on the chronic liver fibrosis model to verify a synergistic therapeutic effect of the pharmaceutical composition of the present disclosure.

Experimental Method

1. Establishment of Chronic Liver Fibrosis Model Mice Induced by CCl₄

160 SPF-grade male C57BL/6 mice that were 6 to 8 week old and had a body weight of 18 g to 20 g were purchased from Yangzhou University, and adaptively raised in an SPF-grade animal room for one week, with a room temperature of 24±2° C., a humidity of 40% to 60%, and a 12 h light/12 h dark cycle. 140 C57 mice were selected and evenly divided into 14 groups with 10 mice per group, including a model group (M group), a forskolin group (F group), an isoforskolin group (IF group), an asiatic acid group (AA group), an asiaticoside group (AS group), an OA group (OA group), a betulinic acid group (BA group), a forskolin+asiatic acid combined administration group (F+AA group), a forskolin+asiaticoside combined administration group (F+AS group), forskolin+OA combined administration groups (low, medium, and high) (F+OA-1:4, 1:1, and 4:1 groups), a forskolin+betulinic acid combined administration group (F+BA group), and an isoforskolin+OA combined administration group (IF+OA group). Every Thursday and Sunday, all mice each were intraperitoneally injected with a 5% carbon tetrachloride (CCl₄)-containing olive oil solution continuously for 8 weeks at an injection volume of 20 mL/kg to obtain a liver fibrosis model caused by chronic liver damage.

2. Treatment Experiment of Chronic Liver Fibrosis Model Mice Induced by CCl₄

Administration Dose

| Group | Dose (mg/kg) |
|---|---|
| M | — |
| F | 20 |
| IF | |
| AA | |
| AS | |
| OA | |
| BA | |
| F + AA | 10 + 10 |
| F + AS | |
| F + OA-1:4 | 4 + 16 |
| F + OA-1:1 | 10 + 10 |
| F + OA-4:1 | 16 + 4 |
| F + BA | 10 + 10 |
| IF + OA | |

Administration mode: A drug was prepared with a 0.5% CMC-Na solution as a solvent into a suspension, and the suspension was intragastrically administered once a day continuously for 8 weeks.

Sampling and Sample Storage:

The mice were administered with a drug continuously for 8 weeks, and after the last administration, the mice were fasted without water deprivation for 12 h; a body weight of each mouse was measured, eyeballs were removed, blood was collected, and a liver was taken out and weighed; and a part of the liver tissue was stored in 4% PFA, and another part of the liver tissue was stored in liquid nitrogen.

A blood sample was allowed to stand in a 4° C. refrigerator for 4 h and then centrifuged at 3,500 rpm for 10 min to 15 min; and a resulting supernatant was collected and tested by a kit to determine AST and ALT levels. The liver tissue stored in liquid nitrogen was homogenized and then centrifuged, and a resulting supernatant was collected and then tested by an enzyme-linked immunosorbent assay (ELISA) kit to determine a PCIII content. The liver tissue stored in 4% PFA was dehydrated, fixed, sectioned, and stained to obtain H&E-stained and Sirius red-stained slide specimens.

Experimental Results

3. Experimental Results

1) Liver Index

In the liver with fibrosis, there is abnormal proliferation of a connective tissue, and a liver weight/body weight ratio (liver weight/body weight ratio=liver weight/body weight*100%) increases. A therapeutic effect of a drug for liver fibrosis in mice can be evaluated through a liver weight/body weight ratio. Results are shown in FIG. 6. For the liver fibrosis model induced by CCl₄, the forskolin+OA (1:1) combined administration group has the lowest liver index, the isoforskolin+OA combined administration group has a medium liver index higher only than the lowest liver index, and there are significant effects in other combined administration groups, which do not have a significant difference; and a liver weight/body weight ratio of mice in a combined administration group is significantly lower than a liver weight/body weight ratio of mice in a single administration group, and a CI value of a liver index reduction rate is greater than 1. At a same dose, the single administration can significantly reduce a liver weight/body weight ratio, and an effect of reducing a liver weight/body weight ratio in a combined administration group is stronger than an effect of reducing a liver weight/body weight ratio in a single administration group, indicating that the combined use of forskolin-isoforskolin with a pentacyclic triterpenoid has a synergistic liver fibrosis improvement effect.

2) PCIII Content

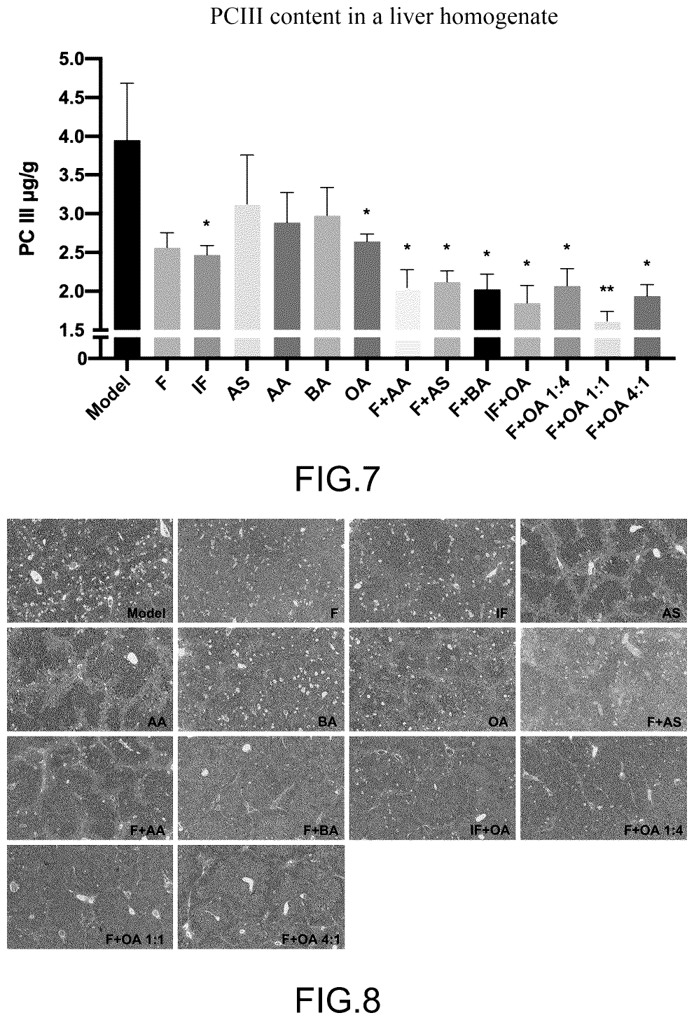

A PCIII content in the liver with fibrosis significantly increases. A PCIII content in the mouse liver can be detected by an ELISA kit to evaluate preventive and therapeutic effects of a drug for liver fibrosis. Results are shown in FIG. 7. For the liver fibrosis model induced by CCl₄, the forskolin+OA (1:1) combined administration group has the lowest hepatic PCIII content, the isoforskolin+OA combined administration group has a medium hepatic PCIII content higher only than the lowest hepatic PCIII content, and there are significant effects in other combined administration groups, which do not have a significant difference; and a hepatic PCIII content of mice in a combined administration group is significantly lower than a hepatic PCIII content of mice in a single administration group, and a CI value of a hepatic PCIII reduction rate is greater than 1. At a same dose, the single administration can reduce a hepatic PCIII content to some degree, and an effect of reducing a hepatic PCIII content in a combined administration group is stronger than an effect of reducing a hepatic PCIII content in a single administration group, indicating that the combined use of forskolin-isoforskolin with a pentacyclic triterpenoid has a synergistic effect.

3) Liver Tissue Sectioning and Quantification

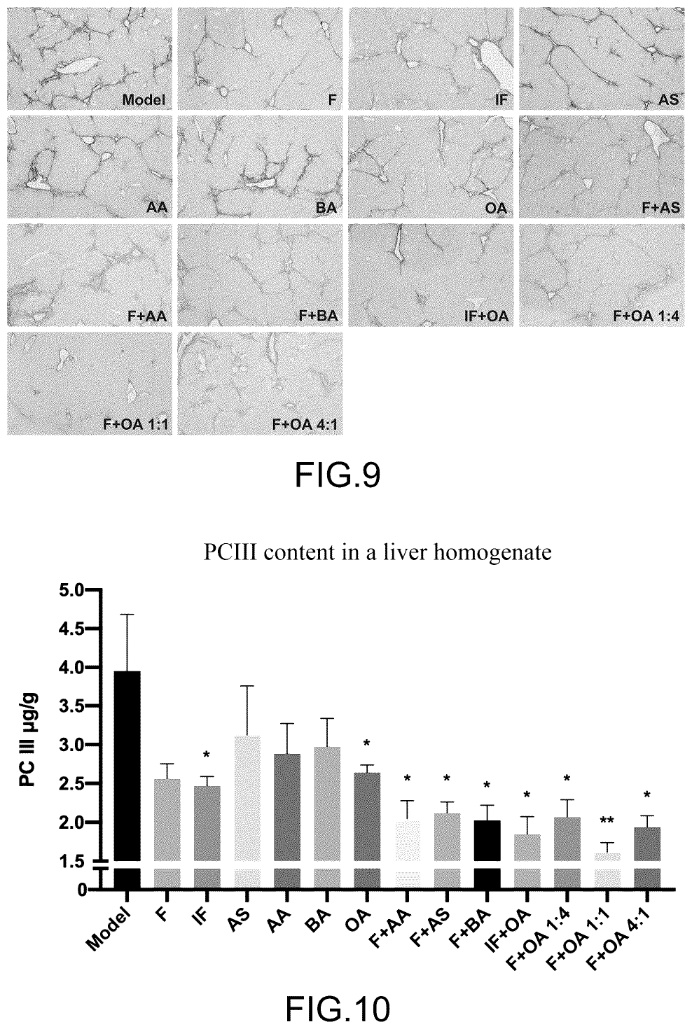

The liver of a liver fibrosis mouse was subjected to H&E sectioning, and the morphology of liver cells was observed to evaluate degrees of inflammation and necrosis (results are shown in FIG. 8); and the liver of the liver fibrosis mouse was subjected to Sirius red staining (Sirius red can specifically stain a fiber component (collagen) red and stain cytoplasm yellow) sectioning (results are shown in FIG. 9), and an area stained red was quantified (results are shown in FIG. 10). For the CCl₄-induced liver fibrosis model, in the forskolin+OA (1:1) combined administration group, the morphology of liver cells is the most complete, there is little inflammatory cell infiltration, and an area stained red is the smallest; in the remaining combined administration groups, the morphology of liver cells is relatively complete, and an area stained red is relatively small; and a therapeutic effect in a combined administration group is significantly better than a therapeutic effect in a single administration group, indicating that the combined use of drugs has a synergistic effect of treating liver fibrosis. At a same dose, the single administration can partially alleviate the liver fibrosis, and an effect of improving the morphology of liver cells, reducing the inflammation and necrosis, and alleviating the liver fibrosis in a combined administration group is stronger than an effect of improving the morphology of liver cells, reducing the inflammation and necrosis, and alleviating the liver fibrosis in a single administration group, indicating that the combined use of forskolin-isoforskolin with a pentacyclic triterpenoid has a synergistic effect.

4) Serum AST and ALT Levels

Common indexes to evaluate liver functions include AST and ALT levels, and AST and ALT levels in serum of mice can be detected by a corresponding kit to evaluate preventive and therapeutic effects of each administration for liver damage after modeling. Results are shown in FIG. 3. For the liver fibrosis model induced by CCl₄, the forskolin+asiaticoside combined administration group has the lowest serum AST and ALT contents, and there are significant serum AST and ALT reduction effects in other combined administration groups, which do not have a significant difference; and serum AST and ALT contents in a combined administration group are significantly lower than serum AST and ALT contents in a single administration group, and CI values of serum AST and ALT reduction rates are greater than 1. At a same dose, the single administration group has a limited effect of reducing serum AST and ALT contents, and an effect of reducing serum AST and ALT contents of mice in a combined administration group is stronger than an effect of reducing serum AST and ALT contents of mice in a single administration group, indicating that the combined use of forskolin-isoforskolin with a pentacyclic triterpenoid has a synergistic liver function protection effect.

Table 3 Impact of each administration on serum AST and ALT contents in liver fibrosis model mice (U/L, x̅±s)

| Group | AST | ALT |
|---|---|---|
| M | 582 ± 32 | 1170 ± 43 |
| F | 447 ± 31 | 922 ± 32 |
| IF | 436 ± 43 | 912 ± 42 |
| AA | 399 ± 22* | 815 ± 53* |
| AS | 406 ± 63* | 882 ± 25 |
| OA | 436 ± 51 | 888 ± 53 |
| BA | 422 ± 45* | 849 ± 34 |
| F + AA | 292 ± 36*** | 484 ± 13*** |
| F + AS | 316 ± 44** | 500 ± 23** |
| F + BA | 315 ± 33** | 573 ± 34** |
| IF + OA | 326 ± 38** | 551 ± 26** |
| F + OA 1:4 | 319 ± 35** | 530 ± 23** |
| F + OA 1:1 | 301 ± 14*** | 514 ± 31** |
| F + OA 4:1 | 315 ± 16** | 543 ± 22** |

The implementations of the present disclosure are described above. However, the present disclosure is not limited to the above implementations. Any modifications, equivalent substitutions, improvements, or the like made within the spirit and scope of the present disclosure should be included within the protection scope of the present disclosure.

1. A pharmaceutical composition, comprising only a first component and a second component as active ingredients, wherein the first component is selected from the group consisting of forskolin, isoforskolin, and a pharmaceutically acceptable salt thereof; and the second component is selected from the group consisting of a pentacyclic triterpenoid and a pharmaceutically acceptable salt thereof;
the pentacyclic triterpenoid is selected from the group consisting of oleanolic acid, asiatic acid, madecassic acid, asiaticoside, madecassoside, and betulinic acid; and
a weight ratio of the first component to the second component is (50:1) to (1:50).

2. (canceled)

3. (canceled)

4. (canceled)

5. The pharmaceutical composition according to claim 1, wherein the weight ratio of the first component to the second component is (20:1) to (1:20).

6. The pharmaceutical composition according claim 1, wherein the pharmaceutical composition further comprises a pharmaceutically acceptable adjuvant; and the pharmaceutically acceptable adjuvant is selected from the group consisting of an excipient, a binder, a disintegrating agent, an emulsifying agent, a preservative, a suspending agent, a fragrance, a pH adjusting agent, a flocculating agent and a deflocculating agent, a surfactant, a filler, a lubricant, a thickening agent, a humectant, a plasticizer, a bacteriostatic agent, a coating material, a foaming agent, a defoaming agent, an encapsulation agent, an isoosmotic adjusting agent, and a stabilizing agent.

7. A formulation comprising the pharmaceutical composition according to claim 1, wherein a dosage form of the formulation is selected from the group consisting of an oral dosage form and an injection.

8. A use of the pharmaceutical composition according to claim 1 in preparation of a drug for preventing, treating, or improving a metabolic disease, a fibrotic disease, and a liver disease.

9. The use according to claim 8, wherein the metabolic disease, the fibrotic disease, and the liver disease are selected from the group consisting of hyperglycaemia, hypertriglyceridemia, hypercholesterolemia, diabetes, obesity, excess visceral fat accumulation, cardiovascular disease, atherosclerosis, liver fibrosis, renal fibrosis, pulmonary fibrosis, skin fibrosis, cardiac fibrosis, fatty liver, liver damage, liver cirrhosis, chronic hepatitis, liver cancer, cholestasis, and cholelithiasis.

10. The use according to claim 8, wherein the metabolic disease, the fibrotic disease, and the liver disease are selected from the group consisting of obesity, non-alcoholic fatty liver disease, liver damage, and liver fibrosis.

11. The pharmaceutical composition according claim 5, wherein the pharmaceutical composition further comprises a pharmaceutically acceptable adjuvant; and the pharmaceutically acceptable adjuvant is selected from the group consisting of an excipient, a binder, a disintegrating agent, an emulsifying agent, a preservative, a suspending agent, a fragrance, a pH adjusting agent, a flocculating agent and a deflocculating agent, a surfactant, a filler, a lubricant, a thickening agent, a humectant, a plasticizer, a bacteriostatic agent, a coating material, a foaming agent, a defoaming agent, an encapsulation agent, an isoosmotic adjusting agent, and a stabilizing agent.

12. A formulation comprising the pharmaceutical composition according claim 5, wherein a dosage form of the formulation is selected from the group consisting of an oral dosage form and an injection.

13. A formulation comprising the pharmaceutical composition according claim 6, wherein a dosage form of the formulation is selected from the group consisting of an oral dosage form and an injection.

14. A formulation comprising the pharmaceutical composition according to claim 1, wherein a dosage form of the formulation is selected from the group consisting of a tablet, a nebulizer, a gel, a granule, a pill, a capsule, a dropping pill, and a suspension.

15. A formulation comprising the pharmaceutical composition according to claim 5, wherein a dosage form of the formulation is selected from the group consisting of a tablet, a nebulizer, a gel, a granule, a pill, a capsule, a dropping pill, and a suspension.

16. A formulation comprising the pharmaceutical composition according to claim 6, wherein a dosage form of the formulation is selected from the group consisting of a tablet, a nebulizer, a gel, a granule, a pill, a capsule, a dropping pill, and a suspension.

17. A use of the pharmaceutical composition according to claim 5 in preparation of a drug for preventing, treating, or improving a metabolic disease, a fibrotic disease, and a liver disease.

18. The use according to claim 17, wherein the metabolic disease, the fibrotic disease, and the liver disease are selected from the group consisting of hyperglycaemia, hypertriglyceridemia, hypercholesterolemia, diabetes, obesity, excess visceral fat accumulation, cardiovascular disease, atherosclerosis, liver fibrosis, renal fibrosis, pulmonary fibrosis, skin fibrosis, cardiac fibrosis, fatty liver, liver damage, liver cirrhosis, chronic hepatitis, liver cancer, cholestasis, and cholelithiasis.

19. The use according to claim 17, wherein the metabolic disease, the fibrotic disease, and the liver disease are selected from the group consisting of obesity, non-alcoholic fatty liver disease, liver damage, and liver fibrosis.

20. A use of the pharmaceutical composition according to claim 6 in preparation of a drug for preventing, treating, or improving a metabolic disease, a fibrotic disease, and a liver disease.

21. The use according to claim 20, wherein the metabolic disease, the fibrotic disease, and the liver disease are selected from the group consisting of hyperglycaemia, hypertriglyceridemia, hypercholesterolemia, diabetes, obesity, excess visceral fat accumulation, cardiovascular disease, atherosclerosis, liver fibrosis, renal fibrosis, pulmonary fibrosis, skin fibrosis, cardiac fibrosis, fatty liver, liver damage, liver cirrhosis, chronic hepatitis, liver cancer, cholestasis, and cholelithiasis.

22. The use according to claim 20, wherein the metabolic disease, the fibrotic disease, and the liver disease are selected from the group consisting of obesity, non-alcoholic fatty liver disease, liver damage, and liver fibrosis.
